(19) United States
(12) Patent Application Publication
(10) Pub. No.: US 2013/0243646 A1
(43) Pub. Date: Sep. 19, 2013

(54) METHOD AND APPARATUS FOR SANITIZING SPORTS IMPLEMENTS AND BALLS

(71) Applicants: Timothy A. Kearns, Emporia, KS (US); J. Thomas Webb, Olathe, KS (US); Ron Brunkow, Americus, KS (US); Ladi O. Shogbamimu, Leawood, KS (US)

(72) Inventors: Timothy A. Kearns, Emporia, KS (US); J. Thomas Webb, Olathe, KS (US); Ron Brunkow, Americus, KS (US); Ladi O. Shogbamimu, Leawood, KS (US)

(21) Appl. No.: 13/424,261

(22) Filed: Mar. 19, 2012

(51) Int. Cl. A61L 2/10

(52) U.S. Cl. CPC A61L 2/10
USPC 422/24; 250/455.11

(57) ABSTRACT

The present invention relates to team sports and sports implements, and more specifically to methods and devices for sanitizing balls during play. One or more sports balls are may be introduced into the device of the present invention which comprises one or more ultra-violet light sources, and has an insertion and a dispensing region, between which each of said plurality of balls travels at a rate that ensures that each of said plurality of balls remains in said apparatus for at least a kill-time period. Ultra violet rays are presented to the surfaces of the plurality of sports balls as the ball traverses between the insertion and extraction regions of the device.

### CROSS-REFERENCE TO RELATED APPLICATIONS

None.

### FIELD OF THE INVENTION

The present invention relates generally to team sports and sports implements, and more specifically to methods and devices for sanitizing balls for sporting events.

### BACKGROUND OF THE INVENTION

People remain concerned on varying levels about the transmission of germs from one person to another. During a team sporting event such as basketball, the ball is passed back and forth between players and officials alike. A plethora of communicable diseases are transmitted by contact between persons or between persons and surfaces which have previously been infected by another person. It is understood that there is a wide variety and seriousness of communicable diseases. Both viral and bacterial varieties of diseases can be spread by contact. There are a number of campaigns and programs to encourage people to wash their hands and/or utilize sanitary wipes, in order to cut-down on and reduce the incidents of the transfer of germs. Anti-bacterial soaps and other biocide agents are also in widespread use today, so as to diminish and combat the spread of diseases.

Despite all of this offensive on disease and the spread through contact with surfaces, the sports environment is one in which there has been little if anything done to address the spread of germs. The industry has focused on disease transmission that may result from players coming in contact with the blood of another player. As such, there are rules and practices in most sports to address situations where blood from one player may come in contact with other players. In some instances the playing surface e.g. the court, or the ball, is wiped down with a sanitizing agent. Mean while some of the more common and non blood borne viruses and bacteria are freely transmitted from players to ball surfaces and then on to other players, as there is no mandated or conducted wipe-down (sanitizing) of balls during the ordinary course of a game. Some of the more common culprit microorganisms are the influenza virus, Norovirus, MRSA, and the common cold rhinovirus.

There are a variety of reasons why sanitization is not conducted on an ongoing basis. Such reasons for example might include the issue of the time delay or interruptions, among other things, or even just the issue of who performs such sanitization i.e. game officials, ball boys/girls, etc. As such, nothing is currently done to address the issue. Consider the game of basket ball at the elementary school level and all the way through the professional level. Players and referees freely contaminate one another with each handling of the ball. All of the associated potential and concerns for wide spread outbreaks or even pandemics of communicable diseases that may arise from the mutations of viruses, along with the increased resistance of bacterial strains to conventional antibiotics, bolster the need to address this problem.

What is needed is a system and method for combating the spread of diseases that may be contracted via physical contact with infected sources. One approach is to sanitize the ball during play, to thereby reduce the presence of contaminants thereon and thereby reduce the risks and instances of exposure of players and officials alike, to surface borne viruses and bacteria. It is known to apply chemical sanitizers in the form of liquids, sprays or powders, to surfaces in an attempt to kill germs. Such applications of sanitizers are not well suited for use in the sports world, primarily because such applications will alter the ball surface and consequently interfere with player ball-handling.

A non-disruptive means for sanitizing a plurality of sports implements is required. The present invention addresses this need and overcomes known devices and methods which are subject to various drawbacks, compromises, inefficiencies, costs or exposure of users to germs and viruses. The present invention addresses this need and overcomes the limitations of the prior art.

### SUMMARY OF THE INVENTION

To address these and other concerns, the present invention provides, among other things, a method and apparatus for sanitizing one or more sports balls wherein an insertion region, a sanitizing cavity and a dispensing region are provided. One or more balls travel within a sanitization cavity or are otherwise maintained within the sanitizing cavity for a kill-period time. One aspect of the system and method of the present invention is to ensure that each of said plurality of balls remains in said apparatus for at least the kill-time period. One or more ultra-violet light sources are disposed within the apparatus such as to present illumination rays to the surface portions of the plurality of sports balls or implements. In another aspect of the present invention, a ball support means for retaining and/or propelling each ball introduced into said apparatus is provided. In a further aspect, the support means is adapted to move each ball rotatably through various axes, as said ball travels between an insertion and a dispensing region, to thereby enhance the exposure of the ball surface to said illumination rays.

### BRIEF DESCRIPTION OF THE DRAWINGS

The present invention is further described with reference to the accompanying drawings, which show various constructions and implementations of the present invention. However, it should be noted that the invention as disclosed in the accompanying drawings is illustrated by way of example only. The various elements and combinations of elements described below and illustrated in the drawings can be arranged and organized differently to result in constructions which are still within the spirit and scope of the present invention. In which,

FIG. 1 is a perspective view of an exemplary embodiment of the present invention illustrating the exterior advertising surfaces;

FIG. 2A is a side view of an exemplary frame rack in an embodiment of the present invention;

FIG. 2B is the top view of the frame rack of FIG. 2A;

FIG. 2C is an illustration of a vertical support of the frame rack of FIG. 2A;

FIG. 2D is an illustration of horizontal arm members in the frame rack of FIG. 2A;

FIG. 2E is an illustration of a screed utilized in the frame rack of FIG. 2A;

FIG. 3 is a perspective view of the frame rack of FIG. 2A, including a depiction of the operation of the rack;

FIG. 4A is transparent view of an exemplary embodiment of the present invention;

FIG. 4B is a left side view of the exemplary embodiment of FIG. 4A; and

FIG. 4C is a top view of the exemplary embodiment of FIG. 4A.

### DETAILED DESCRIPTION

The present invention relates to a system and method for sanitizing team sport balls and sports implements, to thereby mitigate or prevent the spread of diseases communicated through human contact with an infected surface of the balls or sports implements. The present invention provides significant microbiological eradication. The present invention may be implemented and applied to in a wide variety of formats to provide a surface that is relatively free of infectious agents. While the present invention is described with reference to the sport of basketball, it is contemplated that the present system and method along with the teachings herein are equally applicable to other sports, applications and environments, without departing from the spirit and scope of the invention. Additionally, balls of varying sizes such as baseballs, volleyballs, etc and those of varying shapes such as footballs, could also be sanitized utilizing the system and method of the present invention.

Other sports, facilities or applications will benefit from the sanitary and dispensing process disclosed herein. Automated systems that employ mechanical and/or electrical movement of one or more sporting items through a sanitizing chamber to maximize the exposure of the surfaces of said sporting items to one or more sanitization sources, are contemplated and within the scope the present invention.

A key component of the present invention is the movement of an item and its surfaces past one or more sources of ultra-violet (UV) radiation within a sanitization cavity, to periodically and repeatedly expose the surfaces of the item for sanitization. The sanitization/disinfecting effect on an item's surface is a function of the length of time the surface(s) is exposed and the proximity of the surface to the UV light source—intensity. UV light having a wavelength of 200 to 265 nanometers may be utilized for this purpose. However, other varieties of UV light types and characteristics may be utilized without departing from the spirit and scope of the present invention. The intensity of this light is measured in microwatts per square centimeter, and the time is measured in seconds, thus a dose is reported in units of micro-watt seconds per square centimeter. In operation and in a preferred embodiment of the present invention, the movement of the item(s) that is to be sanitized is continuous. The movement through the sanitization cavity is multidirectional in nature i.e. incorporates rotational and revolving aspects, whereby the item is moved about and relative to varying axis in space to thereby maximize exposure of the regions of the item's surface. All of the movement occurs without user involvement or interaction. Furthermore, this approach enables consistent, repeatable, application and results with respect to the sanitization process. An aspect of the present invention is to expose the ball surface for sufficient time such as to kill bacteria and inactivate viruses. Continuous exposure of the item surface will also inactivate bacterial spores, as well. In a further aspect of an embodiment of the invention the described motion occurs under the natural force of gravity. In a further key aspect, the present invention provides means for limiting user exposure and/or the unexpected diffusion of UV rays beyond the confines of the sanitizing device. The following discussion and the invention will be described in the context of the sport of basketball and a typical basketball court environment. It will be immediately understood, however, that the device may be applied to any number of sports, balls, sport implements or environments.

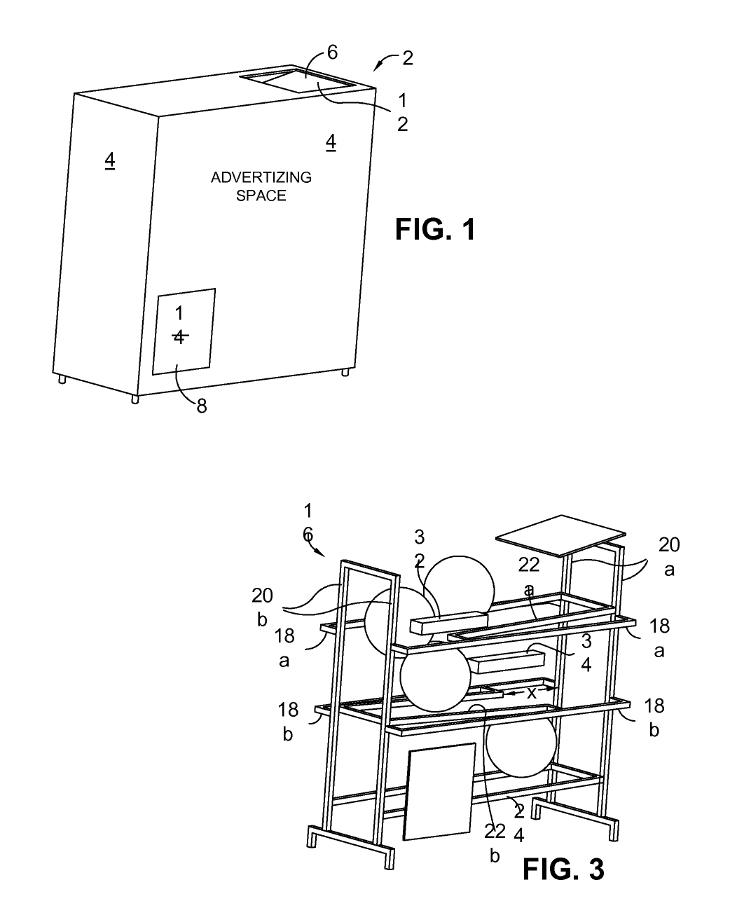

Turning initially to FIG. 1, a perspective view of an embodiment of the present invention is illustrated. The sanitizer apparatus 2 is devised to provide on its exterior surfaces 4, advertisement space(s), which may be illuminated, backlit or otherwise utilized to present advertisements in the form of texts, images, or other depictions. The apparatus 2 may be mobile and or otherwise fixed in place at court side to provide ease of access by officials and a clear view of one or more of its surfaces 4 to spectators. As shown in the illustrated embodiment, the apparatus 2 comprises a sanitization cavity 5 having first and second access apertures 6, 8.

First and second apertures 6, 8 are sized and dimensioned such as to allow an appropriately sized basketball to be passed there-through. First aperture 6 is located and adapted as an insertion location for a basketball 10 (shown in subsequent figures). Second aperture 8 is located and adapted as a receiving or extraction location for the basketball 10. Each of the apertures 6, 8 include a closure or doors 12, 14 respectively. Door 12, 14 are each adapted to be moveable between a closed and opened position.

The door 12 of the first aperture 6 may be spring loaded such that when the door 12 is displaced or otherwise moved into an open position by the insertion of a ball 10, the spring biases the door towards a closed position. In the closed position, the door is coplanar with the adjacent surface of the apparatus 2 in which the aperture 6 is defined. For example, and as shown, when a user places a ball on the door 12, the door is displaced to expose the aperture 6 and allow the ball to pass-through into the apparatus 2. Thereafter, the door 12 springs from the opened position to the closed position.

The door 14 of the second aperture 8 may comprise a knob, handle or other means (not shown) for allowing said door 14 to be moved between an open and closed position. In the open position, access to a ball that is within the apparatus 2 is provided. The door 14 may also be adapted to be biased towards a closed position. The door 14 may be held in a closed position by any number of known methods such as by implementing a friction-fit, a latching engagement, a magnet or any other method known in the art. By utilizing the door 14, a user can access or retrieve a ball 10, by reaching into the apparatus 2. Alternatively, the apparatus may be adapted to simply allow a ball to fall out or be otherwise ejected whenever the door 14 is opened. Door 14 may also be completely absent and instead a retaining member coplanar with aperture 8 may be disposed therein to prevent a ball from simply falling out of the apparatus 2. Another method may incorporate a curtain-like or other assembly of vertical strips of material that provide a covering or other protective isolation of the ball within the apparatus 2. In either of the scenarios respecting aperture 8 and more particularly in the later two scenarios an additional mechanism or implement may be employed to prevent or minimize the exposure of a user's hands or other body parts to UV rays when reaching into the apparatus 2. For example, UV-Opaque materials may be utilized to form a protective shielding around the aperture 8. Alternatively, a switching mechanism may be incorporated adjacent or otherwise, to either or both of the apertures 6,8 of the apparatus 2, whereby the switching mechanism will enable or disable the UV source when either door 12,14 is in the open position. In effect, the present invention is adapted to limit or eliminate UV exposure or ambient UV illumination of the surrounding area of the apparatus 2.

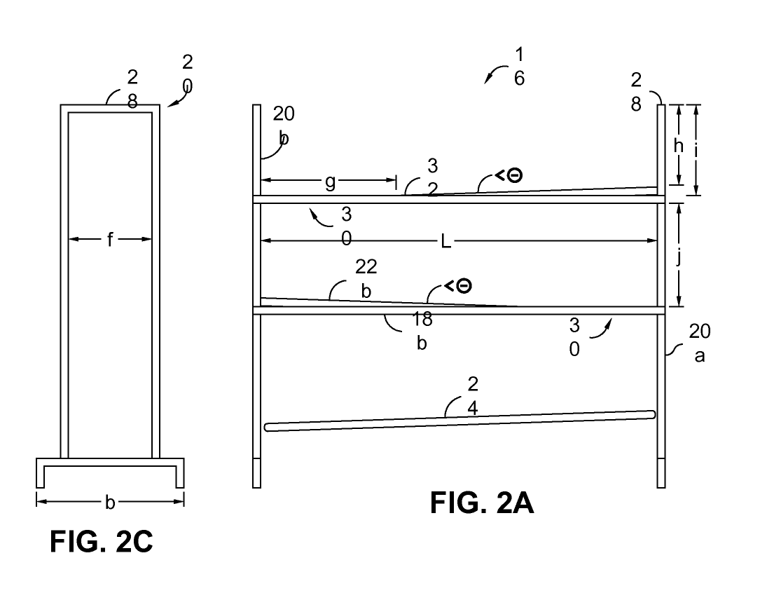

In another aspect of the present invention, a unique and novel construction is provided to move balls between an entry point and exit point while rotating and revolving the balls in multiple directions to thereby maximize the surface area of the ball that is exposed to sanitizing UV rays. This aspect of the invention is best described with reference to FIGS. 2A-2E. The figures display various views and components of an embodiment of the apparatus 2.

Turning first to FIG. 2A, a side view of a rack structure 16 is illustrated. FIG. 2B illustrates a top view of the rack structure 16. The rack structure 16 provides the frame for the apparatus 2, as well as, some component functions of the present invention. Rack structure 16 may be constructed from any of a variety or combination of materials such as wood, steel, aluminum, plastic, etc. Importantly, in a preferred embodiment of the present invention, the rack 16 is constructed such as to move balls through the apparatus 2 by solely utilizing gravity. Alternate embodiments of the invention may employ electro-mechanical means such as a belt driven conveyor system, for moving the balls. In the illustrated embodiment, the rack 16 comprises a frame structure of horizontal arm pairs 18a, 18b (shown in detail in FIG. 2D and collectively designated by the numeral 18); vertical support members 20a, 20b (shown in detail in FIG. 2C collectively designated by the numeral 20); U-ball support screeds 22a, 22b (shown in detail in FIG. 2E collectively designated by the numeral 22); and straight ball support screed 24.

Horizontal arms 18a, 18b are defined have a length c and are affixed to each of the vertical support members 20a, 20b respectively, and define a distance w between the horizontal arm pairs 18a, 18b.

Vertical support member 20 may be a solid piece of material or as in the illustrated example may be constructed with bars or rods that are attached in a rectangular shape having a maximum width f between the longest sides of the member 20, as shown in FIG. 2C. Width f is selected to be less than the diameter of any ball type that would be utilized within the apparatus 2.

The u-ball screed members 22a, 22b are so sized and shaped as to support a ball and guide the motion of the ball along the length of the screeds 22a, 22b. The U-ball support screed members 22a, 22b are u-shaped on a closed end 24 and dog-legged outwardly in opposing directions at an open end 26. The screed 22 defines an inner width f between parallel portions 25 having a length l. A width w2 is defined from end-to-end of the opposed dog-legged portions of the screed 22.

As shown, vertical support members 20a, 20b are connected in opposition to one another, with the horizontal arm pairs 18a, 18b affixed to each support member 20a, and 20b. Horizontal arm pair 18a having a length of c, is disposed in a plane that is a distance j from a distal end of vertical members 20. In one embodiment of the present invention, the distance j is defined such that it is greater than the sum of the diameter of the ball type that will be introduced into the apparatus 2 and a displacement i defined by the location of the u-ball support screed 22a i.e. (j=h+i). Horizontal arm pair 18b is similarly disposed at a distance j from horizontal arm pair 18a. Each of the arm pairs 18a, 18b are affixed to the vertical support members 20a, 20b such as to define a space w1 between each arm pair. Spacing w1 is dimensioned such as to be wider than the diameter of the ball type to be handled by the apparatus 2.

The closed end 24 of the u-ball support screed member 22a is affixed to the inward facing surface of vertical support member 20a at a distance i from the plane of the horizontal arm pair 18a and a distance h from the top end 28 of the vertical support member 20a. The dimension h is selected to be larger than the diameter of the ball type that will be introduced into the apparatus 2. Each of the dog-legged portions of the screed member 22a is affixed to the inward facing surface of each one of the horizontal arm pair 18a. The support screed member 22a is disposed at an acclivity defined by an angle θ between the screed 22a and the horizontal arm pair 18a. U-ball support screed member 22a is disposed such as to cause a ball to roll towards vertical support member 20b from vertical support member 20a. The u-ball support screed member 22a is affixed to the horizontal arm pair 18a and defines a gap 30. Gap 30 has a length g between the inner surface of the vertical support member 20b and a position 32 on the horizontal arm pair 18, where the dog-legged portions of the screed member 22a is affixed. The gap length g is selected to be greater than the diameter of the ball type that will be utilized in the apparatus 2. Importantly, angle θ is predetermined to provide an appropriate rotation/ball movement rate, that will ensure the optimal exposure of the ball surfaces to the UV light source for a required germ/bacterial kill rate.

In another embodiment of the present invention, screed 22 is moveably attached to the vertical support member 20a whereby the screed 22 may be repositioned to alter the angle θ as desired by a user, to thereby further increase or decrease the travel time of a ball within the sanitizing cavity 5.

In a similar yet oppositely affixed and inclined configuration, u-ball screed 22b is affixed to vertical support 20b with an acclivity in the direction of vertical support 20a at the angle θ. Screed 22b is also disposed at distance g from vertical support 20a to define another gap 30.

Straight ball support screed 24 is defined by a pair of elongate sections of framing material, separated by distance f and disposed in a substantially parallel configuration between the vertical support members 20a, 20b. Support screed 24 is affixed to the vertical support members 20a, 20b at an incline that will cause a ball to naturally roll away from vertical support member 20a and towards support member 20b under the force of gravity. Support screed 24 may be disposed at an angle θ and incline with a plane that is substantially parallel the plane of u-ball screed 22a. The framing members of screed 24 are separated by distance f, which as previously described is selected to be smaller than the diameter of the balls that would be used in the apparatus 2. Screeds 22a, 22b and 24 are effectively in a cascading formation.

One or more UV light sources 32, 34 are disposed within the apparatus 2, and may be affixed to the rack structure 16. Importantly, the light sources 32, 34 are located so as to provide optimal surface coverage, of the balls by the UV rays, as the balls move through the apparatus 2, while limiting the opportunity for undesirable exposure to users or the surrounding environment.

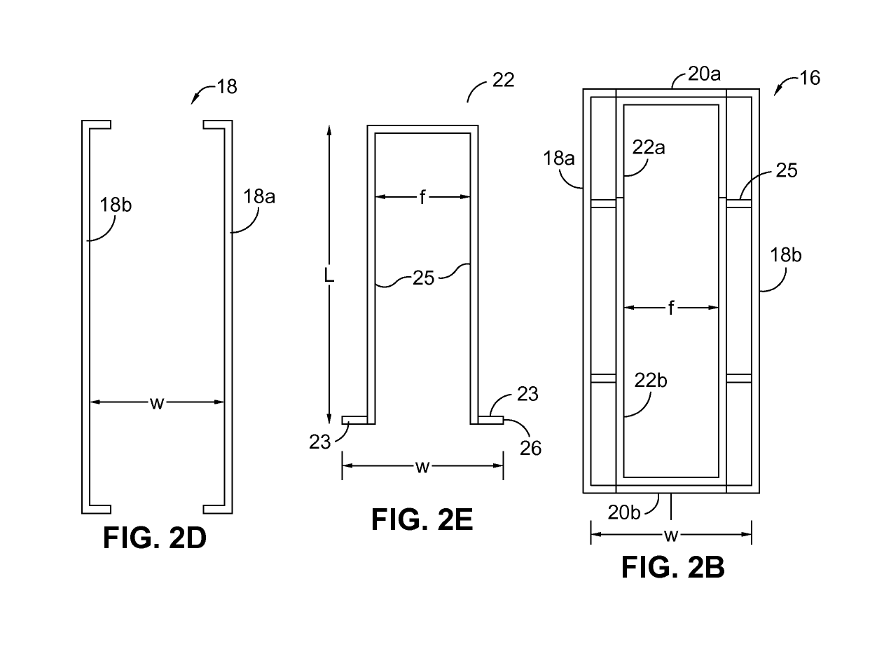

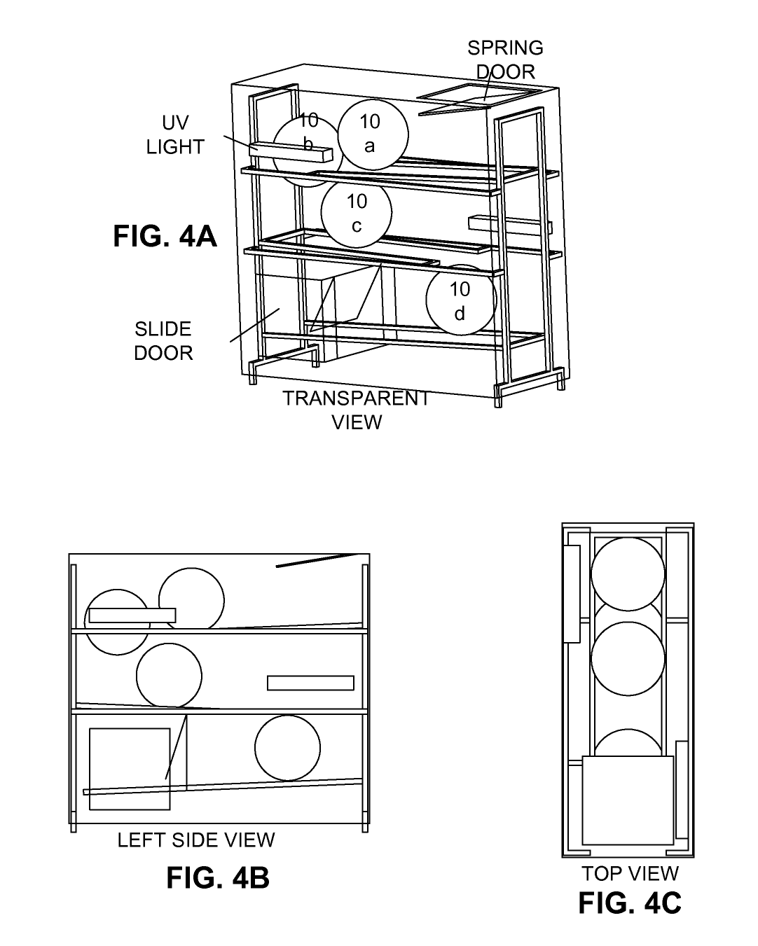

In operation, and as illustrated in FIGS. 3-4, the construction of the rack 16 including the dimensions of the various members and the cascading configuration enable one or more balls 10-10d collectively and/or alternatively referenced herein as ball 10 herein, to be moved through the apparatus 2 and thereby sanitized by the UV light sources 32, 34. Ball 10 is introduced into the apparatus 2 via trap door 6. Ball 10 contacts the screed 22a and rolls under the force of gravity and given the slope angle θ of the screed 22a, in the direction of the acclivity. In the illustrated embodiment, since screed 22a is dimensioned with a separation f between the parallel sides 25 of the screed 22a and the separation f is smaller than the diameter of the ball 10, the ball 10 rolls along the screed 22a towards the vertical support 20b without falling through. When the ball 10 reaches the gap 30, which has a dimension (g by w), wherein both g and w parameters exceed the diameter of ball 10, ball 10 free falls through the gap 30 which is circumscribed by the horizontal arms 18a, the dog-legged ends 23 of the screed 22a, and the vertical support 20b. During the movement and fall, the ball 10 simultaneously rotates about one axis and revolves about another axis, further orienting the ball differently to thereby expose other areas of the ball that may have been obstructed by portions of the screed 22a.

The falling ball 10 ultimately contacts the ball screed 22b located beneath the gap 30. Similar to the actions described respecting the motion of ball 10 along screed 22a, the ball 10 rolls in the direction of the acclivity of screed 22b i.e. towards vertical support 20a, and a gap 31. The gap 31 is circumscribed by the horizontal arms 18b, the dog-legged ends 23 of the screed 22b, and the vertical support 20a. The ball again freely falls through gap 31 while rotating and revolving about two or more axis and finally contacts straight ball screed 24. Straight ball screed 24 is disposed with an acclivity towards vertical support 20b.

The ball 10 rotates in the direction of vertical support pair 20b and comes to rest adjacent vertical support 20b, thereby locating the ball 10 in opposition to aperture 8, through which the ball may be removed from the apparatus 2.

In an alternate embodiment of the present invention, multiple UV sources may be utilized such as to essentially create UV chamber that can effectively illuminate greater surfaces of the ball even prior to ball rotation. A series of vertical blinds or other structure having UV-opaque qualities may be utilized to create an enclosure around the exit aperture 8. Such an enclosure might be adapted to seal off or otherwise conceal the UV chamber of the apparatus 2, after ball has been passed through. Similarly an enclosure or curtain of UV opaque material may also be utilized proximate the entry aperture 6.

In another embodiment of the present invention, the surfaces of each of the screeds 22, 24 may be nodular or otherwise reticulated, to thereby effect a staggered rotational and revolving movement of the ball as it travels along the screed. Such a motion will improve the overall surface exposure of the ball 10 to the UV source. In yet another embodiment, setaceous members may be disposed and aligned along the length of the screeds to introduce friction that may slow the motion of the ball or otherwise force staggered movement of the ball.

In an even further embodiment, the frame members utilized in the construction of the rack 16, may be made from a material that is transparent to UV rays thereby enabling the surfaces of the ball that are in contact with a frame member to simultaneous be radiated by the UV rays as the ball rolls along the screeds 22a, 22b and 24.

In yet another embodiment, the entire apparatus 2 is devised as a cylindrical or elongated cuboid, in which multiple balls may be singly stacked adjacent and in contact with one another.

The cuboid or cylinder having multiple UV light sources disposed therein to sanitize the balls. In this configuration a base plate that is motorized or other rotating means may be utilized to effect rotation and revolution of the multiple balls.

The present invention provides an advantageous apparatus and method, comprising a rack structure, UV lighting and a movement technique that sanitizes sport implements

An exemplary set of features and advantages of an embodiment of the present invention may be described as comprising:
Light source(s) that may be powered by electricity or batteries;
Mechanism for limiting or otherwise eliminating risk of exposure of humans to UV source;
UV light-source with appropriate wavelength to provided an intensity and duration sufficient to kill bacteria and inactivate viruses;
A device structure designed to maximize the exposure of ball surfaces so as to kill bacteria and inactivate viruses on all the surfaces of a ball;
A system with greater reliability than soap, or other disinfectant wipes;
A sanitizer that provides significant microbiological eradication on each pass-through the system; and
Providing consistent, repeatable actions and results due to limited human intervention.

The constructions described above and illustrated in the drawings are presented by way of example only and are not intended to limit the concepts and principles of the present invention. As such, it will be appreciated by one having ordinary skill in the art that various changes in the elements and their configuration and arrangement are possible without departing from the spirit and scope of the present invention as set forth in the claims.

From the foregoing, it will be seen that this invention is one well adapted to attain all the ends and objects hereinabove set forth together with other advantages which are obvious and which are inherent to the method and apparatus. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. As used herein, the terms “having” and/or “including” and other terms of inclusion are terms indicative of inclusion rather than requirement.

1. An apparatus for disinfecting one or more sports ball, the apparatus comprising:
an enclosure;
a rack structure having two or more screeds, each of said screeds affixed to said rack structure at a slope that is defined by a raised end and a low end, said slope having an angle sufficient to have said one or more sports balls move along said screeds under the force of gravity; and
one or more ultraviolet light sources disposed within said enclosure;
said enclosure surrounding said rack structure;
said screeds affixed in an alternating cascading formation wherein adjacent screeds slope in opposite directions;
said enclosure having an insertion aperture and a dispensing aperture, said insertion aperture located at or near the top of said enclosure, and adjacent the raised end of the topmost one of the two or more screeds, and said dispensing aperture located at or near the bottom of said enclosure, and adjacent the low end of the lowest one of the two or more screeds;
(a) said one or more ultra-violet light sources powered and providing ultraviolet rays onto surface portions of the one or more sports balls;
said one or more sports balls traversing rotatably between said insertion location and dispensing location on said rack structure to be thereby sanitized.

2. The apparatus of claim 1 wherein said two or more screeds of said ball rack structure further have reticulated surfaces, to thereby stagger the movement of each ball as it travels along the two or more screeds and thereby revolve the ball about an axis that is transverse to the direction of the ball rotation.

3. The apparatus of claim 1 wherein said ball rack structure is setaceous and frictionally revolves each ball as each ball traverses along said two or more screeds.

4. The apparatus of claim 1 wherein said screeds are formed as aluminum tubes.

5. The apparatus of claim 1 wherein said enclosure is a single formed plastic piece.

6. The apparatus of claim 1 wherein said enclosure has a metal construction.

7. The apparatus of claim 1 wherein said insertion and dispensing apertures are located in positions of said enclosure to maximize the travel time of said balls.

8. The apparatus of claim 7 wherein said insertion and dispensing apertures are located in positions of said enclosure based on the intensity and position of the ultra violet light sources.

9. The apparatus of claim 8 wherein said insertion and dispensing apertures are located in positions of said enclosure that will retain said balls in said apparatus for at least a kill-time period.

10. An method for disinfecting a plurality of sports balls at court side, the method comprising:
providing a sanitization cavity having an insertion aperture and a dispensing aperture between which each of said plurality of balls moves;
providing one or more ultra violet light sources to illuminate said sanitization cavity;
providing a cascading rack for moving said plurality of balls through the sanitization cavity under the force of gravity, said rack configured to maximize exposure of the surfaces of the plurality of balls for at least a kill-time period, whereby said plurality of balls may be disinfected by the illumination of said one or more ultra violet light sources.

11. The method of claim 6 wherein said rack is configured to maximize exposure by the installation of one or more screeds affixed at an acclivity, said acclivity defined by an angle θ wherein said angle θ cause said plurality of balls to be self-propelled through said rack.

12. The method of claim 7 wherein screeds have a reticulated surface to thereby stagger the motion of said plurality of balls.
